(19) United States
(12) Patent Application Publication
(10) Pub. No.: US 2019/0125677 A1
(43) Pub. Date: May 2, 2019

(54) Water-Phase Composition for Producing Microparticles

(71) Applicant: METAL INDUSTRIES RESEARCH & DEVELOPMENT CENTRE, Kaohsiung City (TW)

(72) Inventors: Zong-Hsin Liu, Kaohsiung City (TW); Cheng-Han Hung, Kaohsiung City (TW); Ying-Chieh Lin, Kaohsiung City (TW); Cheng-Tang Pan, Kaohsiung City (TW); Shiao-Wei Kuo, Kaohsiung City (TW); Yao-Kun Huang, Kaohsiung City (TW)

(21) Appl. No.: 15/854,890

(22) Filed: Dec. 27, 2017

(30) Foreign Application Priority Data
Nov. 2, 2017 (TW) 106137972

(51) Int. Cl. A61K 9/16; B01J 13/08

(52) U.S. Cl. CPC A61K 9/1682; A61K 9/1635; B01J 13/08

(57) ABSTRACT

A water-phase composition for producing microparticles includes a water-phase fluid, an amphiphilic polymer stabilizing agent, a water-phase surfactant, and an organic solvent. The amphiphilic polymer stabilizing agent can be polyvinyl alcohol. The water-phase surfactant can be polysorbate 20, polysorbate 80, poloxamer 188, or sodium dodecyl sulfate. The water-phase surfactant can be ethyl acetate, dichloromethane, chloroform, or dimethyl sulfoxide.

### CROSS REFERENCE TO RELATED APPLICATION

The application claims the benefit of Taiwan application serial No. 106137972, filed on Nov. 2, 2017, and the entire contents of which are incorporated herein by reference.

### BACKGROUND OF THE INVENTION

### 1. Field of the Invention

The present invention relates to a water-phase composition and, more particularly, to water-phase composition for producing microparticles.

### 2. Description of the Related Art

Microparticles, also known as microspheres, are spherical particles having a diameter ranging from 1 μm to 1000 μm, are generally used as microcarriers for releasing drug, and have become one of the emerging drug delivery technologies due to the characteristics of targeting, controlled release, stability, and surface modifiability.

Since the diameters of microparticles are small, the first aim is to form microparticles of uniform diameters to make each microparticle have the same drug releasing effect. With reference to FIG. 1, a worker can use a nozzle N to make an oil-phase fluid Fo form a plurality of microdroplets 91 that fall into a tank T receiving a water-phase fluid Fw. The water-phase fluid Fw envelopes each microdroplet 91. Thus, a plurality of microparticle semi-products 92 is formed in the tank T, as shown in FIG. 2. Each microparticle semi-product 92 includes an inner layer 92a formed by the oil-phase fluid Fo and an outer layer 92b formed by the water-phase fluid Fw. Then, the worker can evaporate the outer layer 92b formed by the water-phase fluid Fw, forming products of microparticles only formed by the oil-phase fluid Fo.

However, the surface tension of the oil-phase fluid Fo is smaller than the surface tension of the water-phase fluid Fw (for example, the surface tension of distilled water is 72.75 dyne/cm). Thus, when the microdroplets 91 come into contact with the fluid level of the water-phase fluid Fw, it is likely that only few of the microdroplets 91 penetrate through the fluid level of the water-phase fluid Fw and form the microparticle semi-products 92. Most of the microdroplets 91 would form fiber-like agglomerates that deposit on the fluid level of the water-phase fluid Fw (see FIG. 1) and, thus, cannot form spheres. Improvement is therefore required.

### SUMMARY OF THE INVENTION

To solve the above problem, an objective of the present invention is to provide a water-phase composition for producing microparticles, with the water-phase composition having a surface tension approximating that of the oil-phase fluid.

A water-phase composition for producing microparticles according to the present invention includes a water-phase fluid, an amphiphilic polymer stabilizing agent, a water-phase surfactant, and an organic solvent. In an example, the amphiphilic polymer stabilizing agent is polyvinyl alcohol. The water-phase surfactant can be polysorbate 20, polysorbate 80, poloxamer 188, or sodium dodecyl sulfate. The water-phase surfactant can be ethyl acetate, dichloromethane, chloroform, or dimethyl sulfoxide. Thus, due to addition of the amphiphilic polymer stabilizing agent, the water-phase surfactant, and the organic solvent, the surface tension of the water-phase composition for producing microparticles approximates the surface tension of the oil-phase fluid, such that the plurality of microdroplets fallen into the water-phase composition can be more stably and more uniformly dispersed in the water-phase composition, preventing occurrence of fiber-like agglomerates and increasing formation of spheres from the oil-phase fluid.

In an example, the water-phase fluid is water, the weight of the amphiphilic polymer stabilizing agent is 5-11% of the weight of the water-phase fluid, the weight of the water-phase surfactant is 0.5-1% of the weight of the water-phase fluid, and the weight of the organic solvent is 0.1-10% of the weight of the water-phase fluid. By adjusting the mount of the amphiphilic polymer stabilizing agent, the water-phase surfactant, and the organic solvent, the surface tension of the water-phase composition for producing microparticles more approximates the surface tension of the oil-phase fluid, further increasing formation of spheres from the oil-phase fluid.

The present invention will become clearer in light of the following detailed description of illustrative embodiments of this invention described in connection with the drawings.

### BRIEF DESCRIPTION OF THE DRAWINGS

FIG. 1 is a diagrammatic view illustrating use of a microparticle forming apparatus using a conventional water-phase fluid.

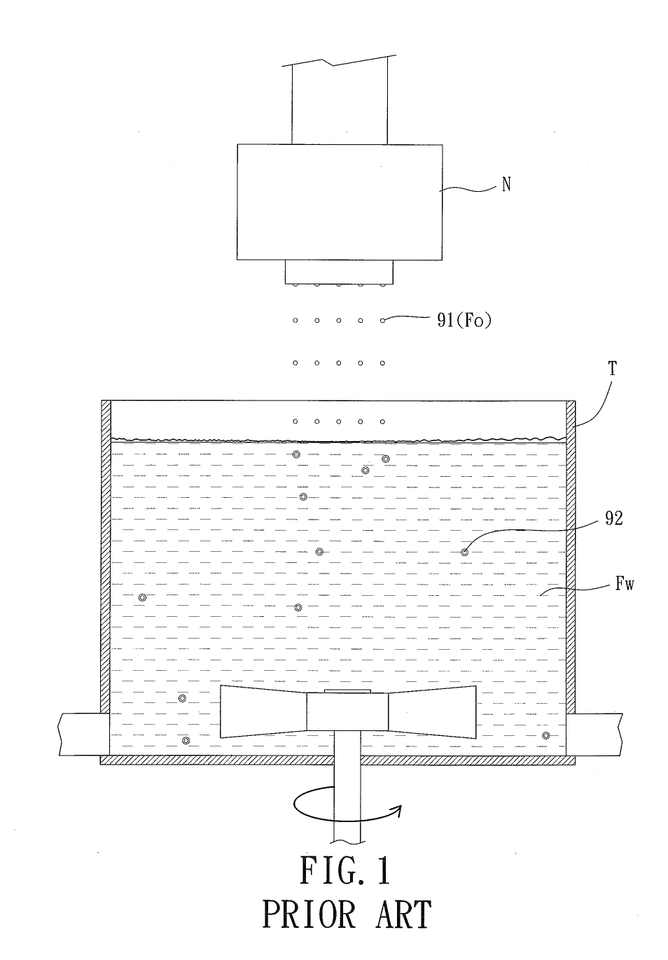

FIG. 2 is a cross sectional view of a microparticle semi-product formed by the microparticle forming apparatus of FIG. 1.

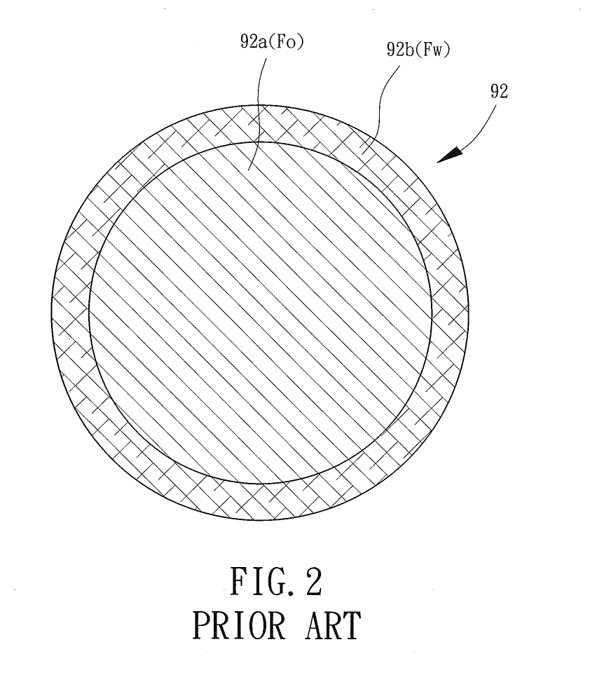

FIG. 3 is a diagrammatic view illustrating use of a microparticle forming apparatus using a water-phase composition for producing microparticles according to the present invention.

FIG. 4 is a cross sectional view of a microparticle semi-product formed by the microparticle forming apparatus of FIG. 3.

### DETAILED DESCRIPTION OF THE INVENTION

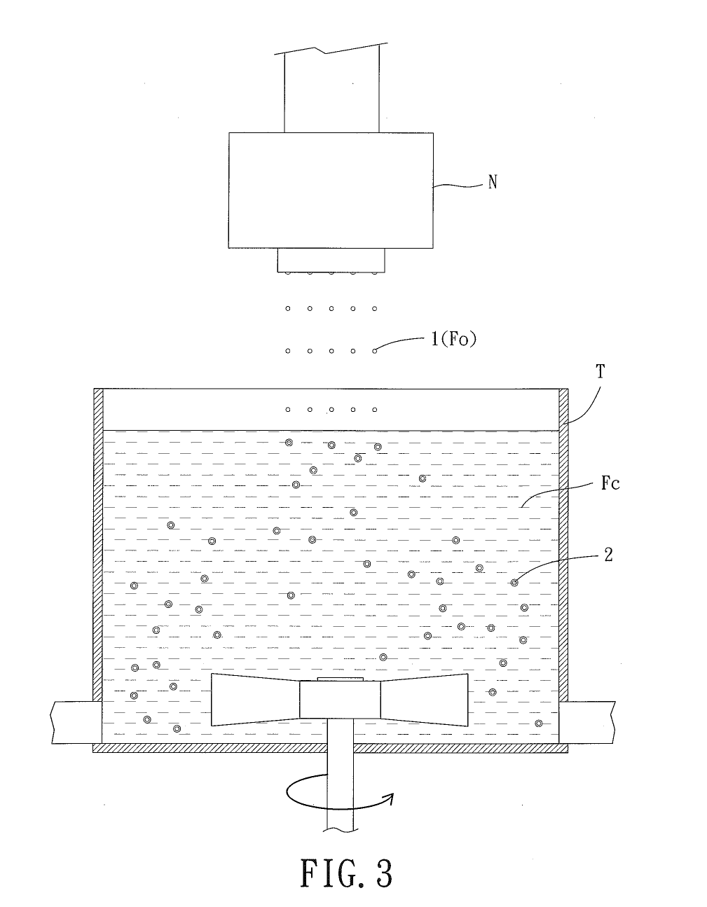

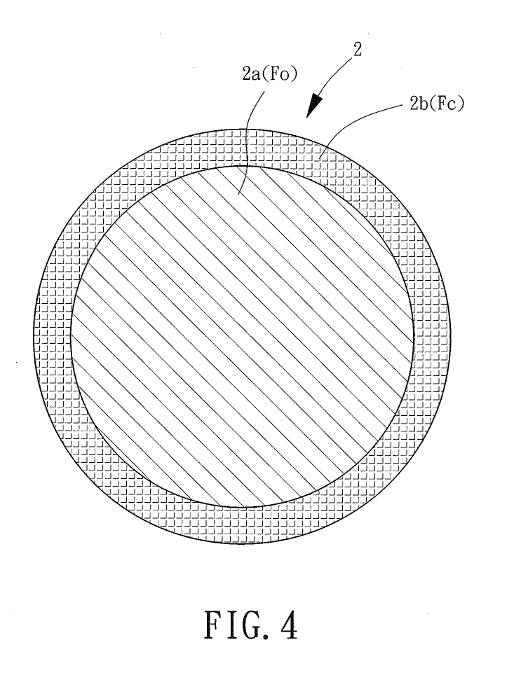

A water-phase composition Fc for producing microparticles of an embodiment according to the present invention includes a water-phase fluid, an amphiphilic polymer stabilizing agent, a water-phase surfactant, and an organic solvent. When a worker uses a microparticle forming apparatus of FIG. 3 to form microparticles, the water-phase composition Fc for producing microparticles can be used to replace the water-phase fluid in the prior art. The water-phase composition Fc is received in a tank T. A plurality of microdroplets 1 formed by an oil-phase fluid Fo falls into the water-phase composition Fc for producing microparticles and is enveloped by the water-phase composition Fc to form a plurality of microparticle semi-products 2 shown in FIG. 4.

In an example, the water-phase fluid is water having a surface tension of 72.75 dyne/cm. By using water as the water-phase fluid, the costs for producing the water-phase composition Fc can be reduced, thereby reducing the costs for producing microparticles.

The amphiphilic polymer stabilizing agent can dissolve in the water-phase fluid and includes a lipophilic segment. When the plurality of microdroplets 1 falls into the water-phase composition Fc for producing microparticles, the amphiphilic polymer stabilizing agent can be stably adhered to the surface of each microdroplet 1 to generate a three-dimensional stabilizing effect, thereby enhancing stable, uniform dispersion of the plurality of microdroplets 1 in the water-phase composition Fc for producing microparticles. In this embodiment, the amphiphilic polymer stabilizing agent is polyvinyl alcohol (PVA). Furthermore, the weight of the amphiphilic polymer stabilizing agent is 5-11% of the weight of the water-phase fluid (i.e., water). Namely, 5-11 g of amphiphilic polymer stabilizing agent is added per 100 g of water.

The water-phase surfactant can dissolve in the water-phase fluid. Furthermore, the water-phase surfactant and the amphiphilic polymer stabilizing agent together reduce the surface tension of the water-phase composition Fc for producing microparticles, which is helpful in smoothly moving the plurality of microdroplets 1 into the water-phase composition Fc for producing microparticles. In this embodiment, the water-phase surfactant is polysorbate 20 (Tween 20, having a HLB value of 16.7), polysorbate 80 (Tween 80, having a HLB value of 15), poloxamer 188 (having a HLB value of 29), or sodium dodecyl sulfate (SDS, having a HLB value of 40). Furthermore, the weight of the amphiphilic polymer stabilizing agent is 0.5-1% of the weight of the water-phase fluid (i.e., water). Namely, 0.5-1 g of water-phase surfactant is added per 100 g of water.

The organic solvent can dissolve in the water-phase fluid. The organic solvent and the amphiphilic polymer stabilizing agent together reduce the surface tension of the water-phase composition Fc for producing microparticles, which is helpful in smoothly moving the plurality of microdroplets 1 into the water-phase composition Fc for producing microparticles. Furthermore, when the oil-phase fluid Fo is falling into the water-phase composition Fc for producing microparticles, the presence of the organic solvent reduces the dispersion speed of the organic solvent (contained in the oil-phase fluid Fo) to be dispersed in the water-phase composition Fc, thereby reducing the occurrence rate of fiber-like agglomerates on the fluid level. In this embodiment, the water-phase surfactant is ethyl acetate, acetone, chloroform, dichloromethane, or dimethyl sulfoxide (DMSO). Furthermore, the weight of the organic solvent is 0.1-10% of the weight of the water-phase fluid (i.e., water). Namely, 0.1-10 g of organic solvent is added per 100 g of water.

With reference to FIG. 3, in production, a worker places the water-phase composition Fc for producing microparticles into the tank T. The oil-phase fluid Fo flows through a nozzle N to form the plurality of microdroplets 1 that falls into the water-phase composition Fc for producing microparticles. Thus, the water-phase composition Fc envelops the plurality of microdroplets 1 to form a plurality of microparticle semi-products 2 shown in FIG. 4. Each microparticle semi-product 2 includes an inner layer 2a formed by the oil-phase fluid Fo and an outer layer 2b formed by the water-phase composition Fc. Then, the worker can obtain microparticles only formed by the oil-phase fluid Fo after filtration, collection, drying, etc.

The water-phase composition Fc for producing microparticles includes the amphiphilic polymer stabilizing agent and the water-phase surfactant to make the plurality of microdroplets 1 smoothly penetrate the fluid level of the water-phase composition Fc into the water-phase composition Fc. Furthermore, since the lipophilic segment of the amphiphilic polymer stabilizing agent generates a three-dimensional stabilizing effect for the plurality of microdroplets 1 fallen into the water-phase composition Fc, formation of spheres from the plurality of microdroplets 1 formed by the oil-phase fluid Fo is increased.

In view of the foregoing, in the water-phase composition Fc for producing microparticles according to the present invention, due to addition of the amphiphilic polymer stabilizing agent, the water-phase surfactant, and the organic solvent, the surface tension of the water-phase composition Fc for producing microparticles approximates the surface tension of the oil-phase fluid Fo, such that the plurality of microdroplets 1 fallen into the water-phase composition Fc can be more stably and more uniformly dispersed in the water-phase composition Fc, preventing occurrence of fiber-like agglomerates and increasing formation of spheres from the oil-phase fluid Fo.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A water-phase composition for producing microparticles comprising a water-phase fluid, an amphiphilic polymer stabilizing agent, a water-phase surfactant, and an organic solvent.

2. The water-phase composition for producing microparticles as claimed in claim 1, wherein the amphiphilic polymer stabilizing agent is polyvinyl alcohol.

3. The water-phase composition for producing microparticles as claimed in claim 2, wherein the water-phase fluid is water, and wherein a weight of the amphiphilic polymer stabilizing agent is 5-11% of a weight of the water-phase fluid.

4. The water-phase composition for producing microparticles as claimed in claim 1, wherein the water-phase surfactant is polysorbate 20, polysorbate 80, poloxamer 188, or sodium dodecyl sulfate.

5. The water-phase composition for producing microparticles as claimed in claim 4, wherein the water-phase fluid is water, and wherein a weight of the water-phase surfactant is 0.5-1% of a weight of the water-phase fluid.

6. The water-phase composition for producing microparticles as claimed in claim 1, wherein the water-phase surfactant is ethyl acetate, dichloromethane, chloroform, or dimethyl sulfoxide.

7. The water-phase composition for producing microparticles as claimed in claim 6, wherein the water-phase fluid is water, and wherein a weight of the organic solvent is 0.1-10% of a weight of the water-phase fluid.
